(19) United States
(12) Patent Application Publication
(10) Pub. No.: US 2007/0185567 A1
(43) Pub. Date: Aug. 9, 2007

(54) CATHETER SYSTEM WITH STENT DEVICE FOR CONNECTING ADJACENT BLOOD VESSELS

(76) Inventors: Richard R. Heuser, Phoenix, AZ (US); James D. Joye, Saratoga, CA (US)

Correspondence Address: KOLISCH HARTWELL, P.C., 200 PACIFIC BUILDING, 520 SW YAMHILL STREET, PORTLAND, OR, 97204, US

(21) Appl. No.: 11/735,609

(22) Filed: Apr. 16, 2007

Related U.S. Application Data
(63) Continuation-in-part of application No. 11/340,324, filed on Jan. 25, 2006.
(60) Provisional application No. 60/887,277, filed on Jan. 30, 2007.

(51) Int. Cl. A61F 2/06

(52) U.S. Cl. 623/1.36

(57) ABSTRACT

A stent assembly is provided for preserving and/or dilating a fistula between a first blood vessel and a second blood vessel. The system comprises an expandable stent having a proximal end, a distal end and a stabilizing device adjacent its distal end. The stabilizing portion may have nominal and active configurations. In the nominal configuration, the stabilizing portion may have a diameter less than the diameter of the first or second blood vessels. In the active configuration, the stabilizing portion may have a diameter substantially equal to the diameter of the second blood vessel, thus preserving the diameter of the second blood vessel. Additionally or alternatively, the stent may be divided into three or more sections, with at least one section exhibiting more robust strength. The section with more strength may be positioned within the fistula, thereby preserving the connection between the first blood vessel and the second blood vessel.

### CROSS REFERENCE TO RELATED APPLICATIONS

This application is a continuation-in-part of application Ser. No. 11/340,324, filed Jan. 25, 2006, the disclosure of which is incorporated by reference. This application also claims the benefit of Provisional Application Ser. No. 60/887,277, filed on Jan. 30, 2007, the disclosure of which is incorporated by reference.

### BACKGROUND

This disclosure relates generally to a catheter system for connecting adjacent blood vessels, e.g, an artery and an adjacent vein to adapt the vein for arterial blood flow. More particularly the disclosure concerns a system of two catheters with mating, magnetic tips for creating openings in the artery wall and vein wall to form a fistula connecting the blood vessels. Further, the disclosure relates to maintaining a fistula between two blood vessels, preserving the diameter of at least one of the vessels and/or the fistula, and allowing safe access of instruments through the vessels and/or the fistula.

A catheter apparatus and method for arterializing a section of a vein to bypass a clogged artery are shown in U.S. Pat. No. 6,464,665, which is hereby incorporated by reference. The method is used to bypass a stenosis in the artery that obstructs blood flow in a portion of the artery. If the obstructed portion of the artery can be bypassed, blood flow will be restored downstream from the stenosis. A vein running alongside the artery in the obstructed portion of the artery can be used for the bypass.

The catheter apparatus includes one catheter for inserting into the artery and another catheter for inserting into the adjacent vein. The physician maneuvers the tips of both catheters to coincident positions within each blood vessel adjacent one end of the obstructed portion of the artery. The physician then creates an opening from the inside of one blood vessel through the vessel wall and then through the wall of the other blood vessel.

An issue here is in co-locating the openings in the two blood vessels and holding the vessel walls in place to ensure that a channel will be created between the vessels so that blood will flow from one vessel to the other. Once a fistula is created between two vessels, stents are often inserted into the fistula and expanded, in order to maintain the fistula. However, veins often have much larger diameters than arteries. Thus, in the case of a single stent with a diameter equal to that of the artery, the portion of the stent that extends into the vein may lack stability in the vein. Further, veins are often more flexible than arteries and hence more prone to collapse. If the vein is collapsed, then a physician may have difficulty in later procedures inserting instruments. Additionally, once a fistula is created between the two vessels, without a way to preserve the connection, the vessels tend to separate.

### SUMMARY OF THE DISCLOSURE

The disclosed system and method provides for creating paired, co-located openings and a consequent fistula between an artery and an adjacent vein to bypass an arterial blockage. The system includes a piercing tool on a first catheter that mates with a receptor on a second catheter to create the co-located openings at one side of the blockage. Magnets incorporated in either or both catheters may be used to draw the piercing tool into the receptor. The piercing tool and receptor typically are provided with complementary, mating contours to draw the piercing tool sufficiently into the receptor to ensure completion of the openings. The openings may be expanded by balloon angioplasty and a stent is typically then installed to interconnect the openings to ensure a fistula is established between the vessels. The process may be repeated at the other side of the arterial blockage to complete the bypass.

An additional aspect of the present disclosure provides for an apparatus that may be used to maintain a fistula between two adjacent blood vessels. The apparatus includes an expandable stent device for inserting into a fistula between two blood vessels. The stent device may have a stabilizing portion configured to preserve the two vessels in close proximity and/or to preserve the diameter of at least one of the vessels. Additionally or alternatively, the stent device may be divided into a plurality of portions, with one portion having more robust radial strength than other portions.

### BRIEF DESCRIPTION OF THE DRAWINGS

FIG. 1 is a partial cross-sectional view showing an obstructed artery, including the obstruction and the area adjacent both ends of the obstruction, and a vein alongside the artery.

FIG. 2 is a cross-sectional view of an embodiment of the present disclosure in the blood vessels of FIG. 1 with a first catheter with a distal end inserted into the artery and a second catheter with a distal end inserted into the vein, the catheters carrying at their distal ends mating tips, i.e., a piercing tool on the first catheter and a receptor on the second catheter.

FIG. 3 is a cross-sectional view of the vein, artery, and two catheters, as in FIG. 2 with the tips of the catheters mated to create a pair of co-located openings in the walls of the vein and artery for connection of a fistula between the artery and the vein.

FIG. 4 is a cross-sectional view of the vein and artery with a balloon inserted through both openings.

FIG. 5 is a cross-sectional view of the vein and artery with a stent installed through the openings between the vein and artery to maintain a fistula therebetween.

FIG. 6 is a cross-sectional view of a first catheter inserted in the artery and a second catheter inserted in the vein at the other end of the obstruction depicted in FIGS. 1-4, the catheters including mating tips shown in a joined position to create a second pair of co-located openings through the vein and artery walls.

FIG. 7 is a cross-sectional view of the vein and artery with a balloon inserted through the second pair of openings between the vein and the artery.

FIG. 8 is a cross-sectional view of the vein and artery with a second stent installed through the second pair of openings between the vein and artery to maintain a fistula therebetween.

FIG. 9 is a close-up perspective view of the mating tips of the first and second catheters, showing the receptor, which includes a proximal end, a distal opening, and a channel providing a guide surface, and the piercing tool, which includes a needle and a plug encompassing the catheter adjacent the base of the needle, and showing the contours of the plug, needle, and receptor channel that provide for mating between the tips.

FIG. 10 is a piercing tool for use in a second embodiment of the present disclosure that includes a base and a needle that is offset from the base by an angle.

FIG. 11 illustrates the use of the piercing tool of FIG. 10 in conjunction with a double-balloon catheter to create openings in a vein and an artery.

FIG. 12 illustrates the use of the piercing tool of FIGS. 2, 3, 6, and 9 in conjunction with a double-balloon catheter to create openings in a vein and an artery.

FIG. 13 shows another aspect of the present disclosure in a constricted configuration being traversed through a blood vessel.

FIG. 14 shows the embodiment of FIG. 13 in an expanded configuration traversing a fistula between two blood vessels.

FIG. 15 shows an example of the stabilizing portion in the active configuration.

FIG. 16 shows another example of the stabilizing portion in the active configuration.

FIG. 17 shows another example device outside of a human body.

FIG. 18 shows the device of FIG. 17 traversing a fistula between two blood vessels.

### DETAILED DESCRIPTION OF THE PREFERRED EMBODIMENTS

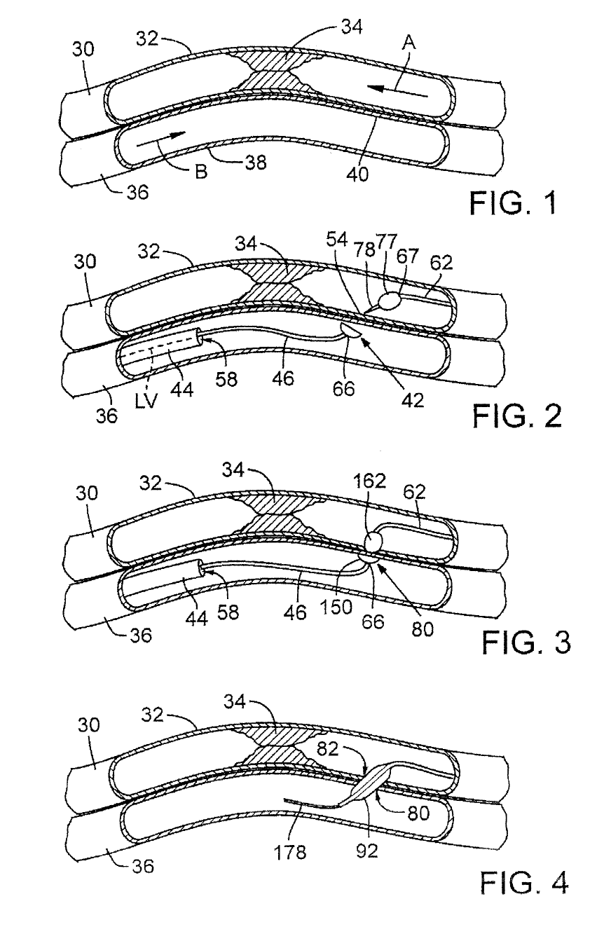

As shown in FIG. 1, an artery 30, formed by an artery wall 32, has a blood flow, indicated by arrow A, that is partially or totally blocked by an obstruction or occlusion 34, typically formed by plaque. A vein 36 roughly similar in dimension to artery 30 lies alongside and generally parallel to artery 30. Vein 36, formed by a vein wall 38, includes, in the area proximal to occlusion 34, a portion 40 in close proximity to artery 30 that the physician has selected as a venous site for creating a fistula between artery 30 and vein 36. The normal blood flow through vein 36 would be in the direction indicated by arrow B.

An embodiment of the disclosed system, indicated generally at 42 in FIG. 2, is a catheter apparatus that includes a first catheter 62 and a second catheter 44. In FIG. 2, the first catheter is in the artery and the second catheter is in the vein, but this can be reversed. Similarly, the first catheter in the artery is shown upstream from occlusion 34, but this may alternatively be reversed to begin the procedure downstream from the occlusion and proceeding afterwards to the upstream side.

Second catheter 44 may include at least one lumen 58 which runs generally parallel to a longitudinal axis LV of catheter 44. A wire 46 may be inserted through lumen 58. Typically, wire 46 has an outer diameter of 0.035-inches, but any suitable dimension may be used. Wire 46 may be controllable by the physician in position relative to catheter 44. Wire 46 may be a guidewire for catheter 44, or a separate guidewire may be used, with other lumens in catheter 44 providing the channel for the separate guidewire.

As shown in FIG. 2, first catheter 62 of catheter apparatus 42 includes a distal end 67 that the physician may insert into artery 30 for positioning adjacent arterial fistula site 54. First catheter 62 may include one or more lumens running generally parallel to a longitudinal axis of catheter 62. First catheter 62 may be guided along a guidewire or may itself be a guidewire, typically with an outer diameter of 0.035-inches, although any suitable dimension may be used. First catheter 62 preferably is hollow.

A piercing tool 77 that includes a sharp needle 78, may be selectively deployed, as shown in FIGS. 2 and 3, or withdrawn into the lumen of catheter 62. Needle 78 is preferably withdrawn while catheter 44 is maneuvered to the fistula site so as not to cause trauma to the blood vessel wall.

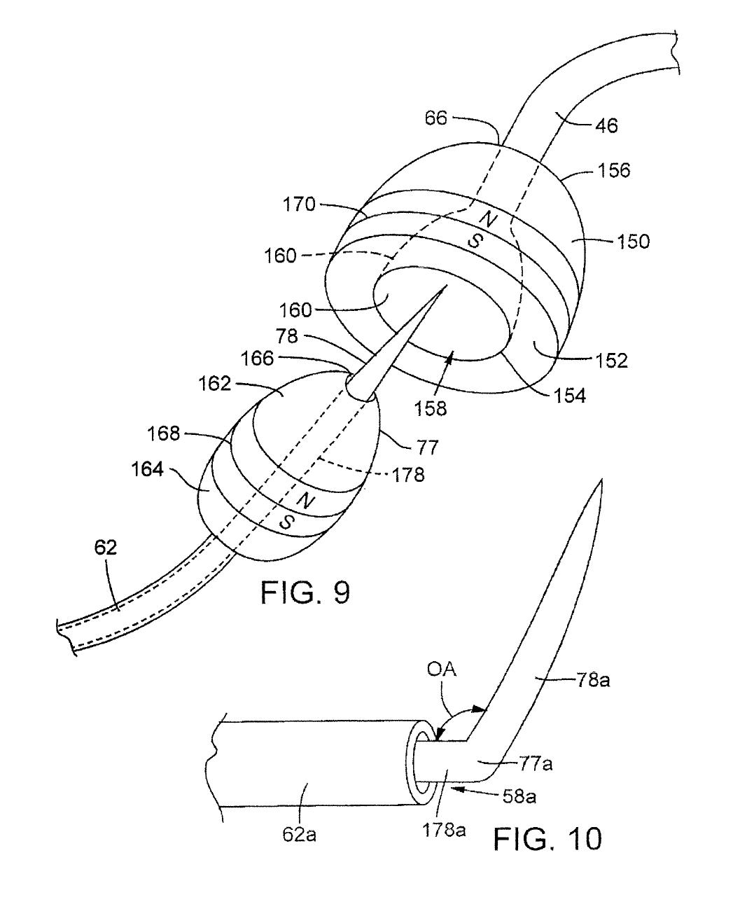

As best seen in FIG. 9, needle 78 may be disposed at the distal end of a wire 178 disposed in the lumen of catheter 62. The physician can control the positioning of wire 178 and needle 78 relative to catheter 62. Guidewire 46 may include a receptor 150, such as substantially cup-shaped socket 152. Receptor 150 includes a distal opening 154, preferably circular, and a proximal end 156. Receptor 150 includes a channel 158 leading from opening 154 toward proximal end 156. Channel 158 preferably narrows in a direction from opening 154 toward proximal end 156. Channel 158 is defined by an inner surface 160 that provides a guide surface for needle 78 that directs the needle toward proximal end 156 of receptor 150. Channel 158 may be substantially conical, or have such other shape as tends to mate with, and guide piercing tool 77 into receptor 150.

Piercing tool 77 on catheter 62 preferably includes a plug 162 provided with an outer contour that narrows from a proximal end 164 toward a distal end 166. Plug 162 preferably mates with channel 158 in receptor 150. Plug 162 preferably encompasses catheter 62 adjacent the distal end of the catheter. As seen in FIGS. 2, 3, and 9, the piercing tool and the receptor have a complementary configuration that supports their mating together.

Typically, piercing tool 77 will include a magnet with one pole oriented toward the distal end of the tool, while receptor 150 will include a magnet with the opposite pole oriented toward the distal end of the receptor which will draw the needle into the receptor. For example, the magnets may be annular rings or donuts and formed of a strong permanent magnet material suitable for the intended use.

A typical arrangement, shown in FIG. 9, is that plug 162 includes a first magnet 168 generally in a donut shape and having a north pole N positioned distally with respect to a south pole S. Typically magnet 168 is spaced from the distal end of plug 162. A second magnet 170 may be disposed on, or form an integral part of receptor 152, preferably adjacent distal opening 154 of socket 152. Second magnet 170 may be arranged with a south pole S distal of a north pole N to attract magnet 168 when the tips of the two catheters are in proximity, e.g., with each catheter in an adjacent blood vessel. Alternatively or in addition one or more magnets may be arranged in various locations on plug 162 and/or needle 78 and on or in receptor 150, e.g., adjacent proximal end 156, with the poles arranged to draw piercing tool 77 into receptor 150.

As shown in FIGS. 3 and 4, after creating openings 80, 82 with a tool such as needle 78, the physician withdraws catheter 62 from the fistula site, leaving wire 178 in place, and a balloon 92 may be inserted over wire 178 and through openings 80, 82 and inflated to enlarge the openings. Balloon 92 may include radiopaque markers and may be inflated with a solution containing a radiopaque dye or contrast to allow the physician to radiographically monitor and adjust the position of the balloon before, during, and after inflation.

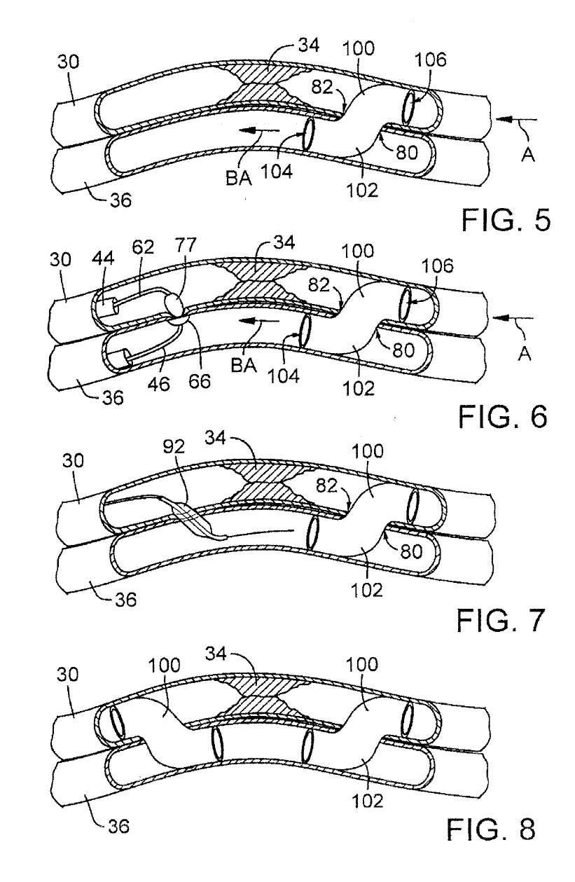

As shown in FIG. 5, a device for maintaining an open, leak-free connection between openings 80 and 82, such as stent 100, is inserted through the openings. Stent 100 includes a frame 102 having two open ends 104 and 106 that preferably create leak-free couplings to the inside of artery 30 and vein 36. With openings 80, 82 connected to form a fistula, vein 36 is arterialized, and blood flows from artery 30 into vein 36 in the direction indicated by arrows A and BA.

Stent 100 is typically a short, covered stent, such as the Hemobahn stent made by WL Gore & Associates.

As shown in FIGS. 6, 7, and 8 a second pair of co-located openings may be created, and a stented fistula established therebetween, using essentially the same catheter system and method as described for FIGS. 1-5 and 9. FIG. 6 illustrates that the first catheter with the piercing tool preferably is inserted into the artery and the openings created from the artery into the vein. Alternatively the openings may be created from the vein into the artery.

An alternative embodiment for the piercing tool in shown in FIG. 10. This tool 77a may be used with a metal guidewire 62a that preferably includes a lumen 58a. An inner wire 178a may be inserted in lumen 58a, providing a base for a needle 78a. The coupling between the needle and base incorporates a curvature such that the needle is nominally offset from the base by an angle OA, typically between about 30-degrees and about 90-degrees. Inner wire 178a is typically made of a sufficiently rigid material, such as nitinol and/or stainless steel, as to maintain the offset angle as the needle is used to pierce blood vessels. Guidewire 62a is preferably formed of a sufficiently rigid material such that when needle 78a is retracted into lumen 58a, the curvature between the needle and the base is overcome and the needle temporarily aligns with the base in a non-traumatic configuration. Inner wire 178a may have an outer diameter of 0.010, 0.014, 0.018, or 0.021-inches, or such other dimension as is suited to the particular application.

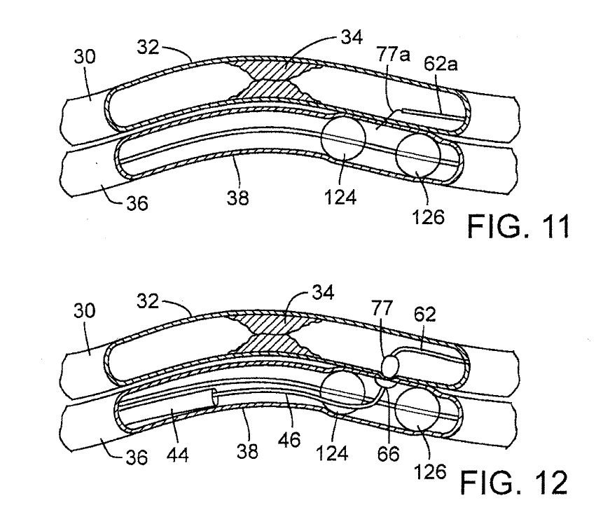

As shown in FIG. 11, piercing tool 77a may be inserted in artery 30, typically while withdrawn into the catheter 62a while maneuvering to the fistula site. Piercing tool 77a may be used in conjunction with a catheter having two balloons 124 and 126 that are inserted in vein 36. In such case, the catheter tips are maneuvered to opposing sides of the proposed fistula site and balloons 124 and 126 are inflated to press the vein wall against the artery wall. Also, fluid may be injected into the sealed-off area to further press the two blood vessel walls together. Then piercing tool 77a is deployed and maneuvered through the artery and then the vein wall to create openings for forming the fistula as for the embodiments described above.

FIG. 11 depicts the piercing tool and the balloon catheter in different vessels. Alternatively, piercing tool 77a may be inserted in the same blood vessel with the balloon catheter. In such an embodiment, the balloons are preferably independently inflatable, and typically the distal balloon 124 is inflated first to stop blood flow. Then, piercing tool 77a is maneuvered to the fistula site in a manner similar to that for the previously described embodiment, typically with the piercing tool withdrawn into the guidewire to the non-traumatic configuration.

With the piercing tool at the fistula site, the proximal balloon 126 is inflated to seal off the fistula site and also to press the vein against the artery. Then, piercing tool 77a is deployed at the end of guidewire 62a and maneuvered by the physician to create the openings from one blood vessel, through both walls, to the other blood vessel.

In either case, piercing tool 77a may be used to create multiple pairs of co-located openings which are then stented to arterialize a portion of the vein to bypass a blockage using a similar method as described above for the embodiment of FIGS. 1-9.

As shown in FIG. 12, the double balloon catheter may also be used in conjunction with the catheters 44 and 62 that include the mating tips. In this embodiment, the double balloon catheter helps to control blood flow at the planned fistula site and to press the blood vessel walls together to assist in the mating of the tips. The fistula creation otherwise proceeds in a similar manner as for the embodiment of FIGS. 1-9.

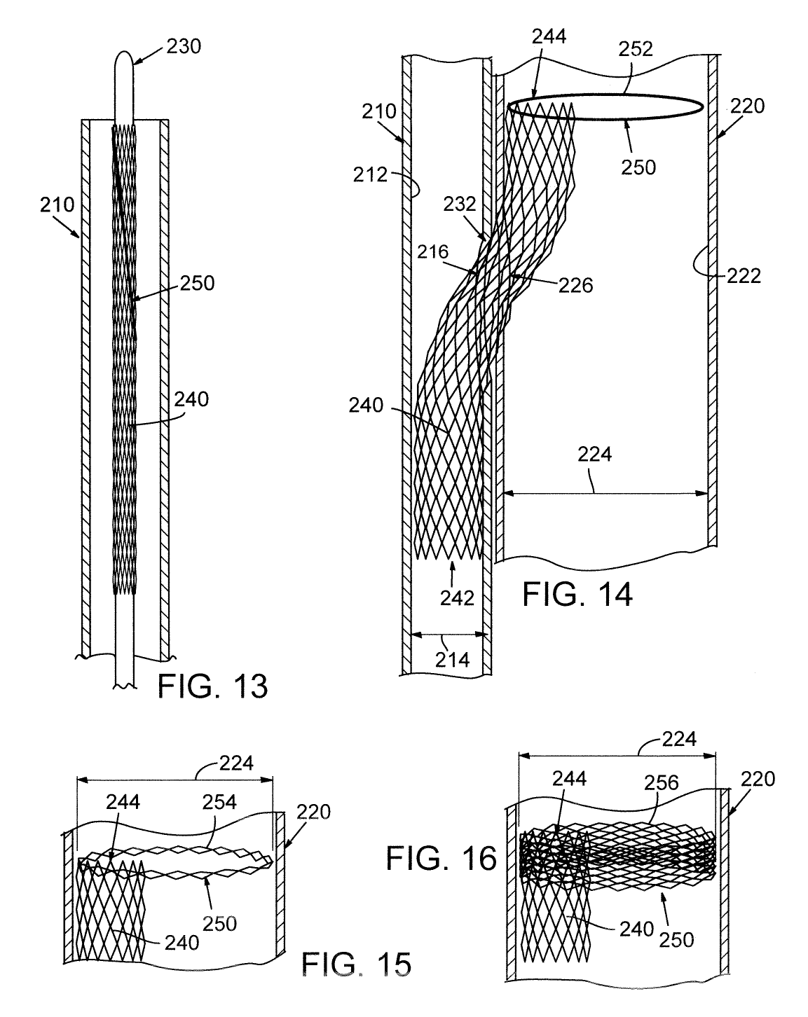

Another aspect of the present disclosure is depicted in FIG. 13, where an embodiment is seen having an expandable tubular portion 240 in a constricted configuration disposed on a catheter 230, the embodiment traversing a first blood vessel 210. The catheter may be inserted to a position within a vessel adjacent to a fistula 232 (shown in FIG. 14).

FIG. 14 depicts the apparatus in an expanded configuration and connecting the first blood vessel 210, formed by vessel wall 212 and having a first diameter 214, with a second blood vessel 220, formed by vessel wall 222 and having a second diameter 224. There is a fistula 232 between the first blood vessel 210 and the second blood vessel 220. The fistula 232 comprises a first opening site 216 in the first vessel 210 and a second opening site 226 in the second blood vessel 220. The fistula 232 may have been created by any known method, including but not limited to the methods recited in U.S. Pat. No. 6,464,665, or the methods described above.

As seen in FIG. 4, the expandable tubular portion 240 has a proximal end 242 which remains in the first blood vessel 210 and a distal end 244 which extends into the second blood vessel 220. A section of the expandable tubular portion 240 between the proximal end 242 and the distal end 244 passes through the fistula 232. A stabilizing portion 250 is disposed adjacent to the distal end 244 of the expandable tubular portion 240, the stabilizing portion 250 having a nominal configuration and an active configuration.

The stabilizing portion 250 in the nominal configuration may have a diameter which is less than the second diameter 224. In the active configuration, the stabilizing portion 250 may have a diameter which is greater than the diameter of the stabilizing portion in the nominal configuration. The diameter of the stabilizing portion 250 in the active configuration may also be substantially equal to the second diameter 224 of a second blood vessel 220.

FIG. 14 depicts the stabilizing portion 250 in the active configuration as a wire hoop 252. The wire hoop 252 may be made of any suitable material. In one embodiment, the wire hoop 252 is metallic. In another embodiment, the wire hoop 252 is made at least partially of nitinol.

FIG. 15 shows an alternative embodiment where the stabilizing portion 250 takes the form of a row of stent cells 254 disposed on the distal end 244 of the tubular portion 240. The row of stent cells 254 is configured so that the first cell is coupled to the last, thus forming a hoop of stent cells. The row of stent cells 254 may be expandable to a diameter substantially equal to the second diameter 224. In one embodiment, the row of stent cells 254 is metallic. In another embodiment, the row of stent cells 254 is made at least partially of nitinol.

FIG. 16 shows another embodiment where the stabilizing portion 250 takes the form of a plurality of rows of stent cells 256 disposed on the distal end 244 of the tubular portion 240. The rows of stent cells 256 are configured so that the first cell in each row is attached to the last cell in each row, thus forming a tube of stent cells. The rows of stent cells 256 may be expandable to a diameter substantially equal to the second diameter 224. In one embodiment, the rows of stent cells 256 are metallic. In another embodiment, the rows of stent cells 256 are made at least partially of nitinol.

It should be understood that the diameter of the stabilizing portion 250 in the active configuration, whether in the form of a wire hoop 252, a row of stent cells 254, or a plurality of rows of stent cells 256, may be slightly larger than the second diameter 224, so that the stabilizing portion 250 stretches the second blood vessel 220 slightly, without damaging the vessel. The diameter of the stabilizing portion 250 in the active configuration may alternatively be equal to or slightly smaller than the second diameter 224.

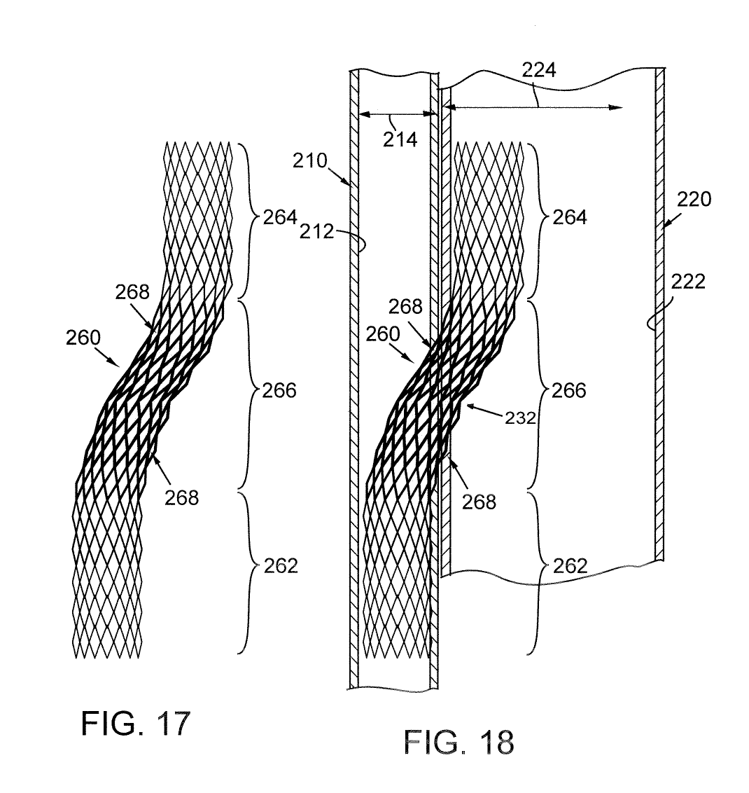

FIG. 17 shows another embodiment of the device outside a living body. This embodiment comprises an expandable tubular stent 260 with a first section 262, a second section 264, and a third section 266. The first 262 and second 264 sections have first and second radial strengths that may or may not be equal to one another. The third section 266 is disposed in between the first 262 and second 264 sections and has a third radial strength that may be greater than the first or second radial strengths. The third section may additionally have one or more bends 268 by default.

FIG. 18 depicts the embodiment shown in FIG. 17 in an expanded configuration connecting a first blood vessel 210 and a second blood vessel 220 via a fistula 232. The first section 262 remains in the first vessel 210, and the second section 264 is extended into the second vessel 220. The third section 266, with its strength being greater than the first or second strengths, is disposed in the fistula 232, thereby holding the two vessels in close proximity and maintaining the diameter of the fistula 232. In its expanded configuration, the tubular stent 260, and particularly the third section 266, may further dilate the diameter of the fistula 232.

The expandable tubular portion 260 may be constructed of any suitable material. In one embodiment, the expandable tubular portion 260 comprises a metallic mesh stent. This embodiment may be used as a placeholder until a more permanent device having an impermeable membrane may be placed in the fistula 232. In another embodiment, the expandable tubular portion 260 is constructed at least partially out of nitinol. In another embodiment, the expandable tubular stent 260 comprises a mesh skeleton with an impermeable membrane (e.g., polytetrafluoroethylene) covering its surface.

In another example, the expandable tubular stent 260 may further include a stabilizing portion 250 similar to the embodiments shown in FIGS. 14-16. The stabilizing portion 250 may be disposed on a distal end of the second section 264, and may take the form of an expandable hoop 252, a row of stent cells 254, or a plurality of rows of stent cells 256, as described above.

It is believed that the following claims particularly point out certain combinations and subcombinations that are directed to one of the disclosed disclosures and are novel and non-obvious. Disclosures embodied in other combinations and subcombinations of features, functions, elements and/or properties may be claimed through amendment of the present claims or presentation of new claims in this or a related application. Such amended or new claims, whether they are directed to a different disclosure or directed to the same disclosure, whether different, broader, narrower or equal in scope to the original claims, are also included within the subject matter of the disclosures of the present disclosure.

We claim:

1. A device for maintaining a fistula between a first blood vessel having a first diameter and a second blood vessel having a second diameter, the device comprising:
an expandable tubular portion having a proximal end and a distal end, the expandable tubular portion insertable to a position wherein it extends from the first vessel through an fistula into the second vessel, with the proximal end in the first blood vessel and the distal end in the second blood vessel;
a stabilizing portion, adjacent to the distal end of the expandable tubular portion, being manipulable between a nominal configuration and an active configuration, the nominal configuration having a third diameter which is less than the second diameter and the active configuration having a fourth diameter that is substantially equal to the second diameter.

2. The device of claim 1, wherein the expandable tubular portion is comprised at least partially of nitinol.

3. The device of claim 1, wherein the stabilizing portion is an expandable hoop.

4. The device of claim 1, wherein the stabilizing portion is a row of stent cells arranged so that a first cell in the row is coupled to a last cell in the row, forming a hoop.

5. The device of claim 1, wherein the stabilizing portion comprises a plurality of rows of stent cells arranged so that a first cell in each row is coupled to a last cell in each row, forming a tube.

6. The device of claim 1, the expandable tubular portion further comprising:
a first section having a first radial strength, a second section having a second radial strength, and a third section, positioned between the first and second sections and having a third radial strength which is stronger than the first or second radial strengths, the third section having at least one bend;
wherein the first section extends through the first blood vessel to a position adjacent to the fistula, the third section extends through the fistula, and the second section extends from a position adjacent the fistula into the second blood vessel.

7. The device of claim 6, wherein the third section has a nominal configuration and an active configuration, the third section in the nominal configuration having a diameter less than that of the fistula and the third section in the active configuration having a diameter substantially equal to that of the fistula.

8. The device of claim 6, wherein the first, second and third sections each have a nominal configuration and an active configuration: the first section in the nominal configuration having a diameter less than the first diameter and the first section in the active configuration having a diameter substantially equal to the first diameter; the second section in the nominal configuration having a diameter less than the second diameter; and the third section in the nominal configuration having a diameter less than the diameter of the fistula and the third section in the active configuration having a diameter substantially equal to the diameter of the fistula.

9. The device of claim 6, wherein the first, second and third sections each have a nominal configuration and an active configuration: the first section in its nominal configuration having a diameter less than the first diameter and the first section in its active configuration having a diameter substantially equal to the first diameter; the second section in the nominal configuration having a diameter less than the second diameter and the second section in the active configuration having a diameter substantially equal to the second diameter; the third section in the nominal configuration having a diameter less than that of the fistula and the third section in the active configuration having a diameter substantially equal to that of the fistula.

10. The device of claim 1, wherein the stabilizing portion is metallic.

11. The device of claim 1, wherein the stabilizing portion is constructed at least partially of nitinol.

12. The device of claim 1, wherein the expandable tubular portion is a mesh stent covered in an impermeable membrane.

13. A device for stabilizing a fistula between a first blood vessel having a first diameter and a second blood vessel having a second diameter, the device comprising:
an expandable tubular stent with a first section having a first radial strength, a second section having a second radial strength, and a third section, positioned between the first and second sections, having a third radial strength which is stronger than the first or second radial strengths, the third section having at least one bend, the expandable tubular stent being insertable to a position wherein the first section extends through the first blood vessel to a position adjacent to the fistula, the third section extends through the fistula, and the second section extends from a position adjacent the fistula into the second blood vessel.

14. The device of claim 13, wherein the third section has a nominal configuration and an active configuration, the third section in the nominal configuration having a diameter less than that of the fistula and the third section in the active configuration having a diameter substantially equal to that of the fistula.

15. The device of claim 13, wherein the first, second and third sections each have a nominal configuration and an active configuration: the first section in the nominal configuration having a diameter less than the first diameter and the first section in the active configuration having a diameter substantially equal to the first diameter; the second section in the nominal configuration having a diameter less than the second diameter; and the third section in the nominal configuration having a diameter less than the diameter of the fistula and the third section in the active configuration having a diameter substantially equal to the diameter of the fistula.

16. The device of claim 13, wherein the first, second and third sections each have a nominal configuration and an active configuration: the first section in its nominal configuration having a diameter less than the first diameter and the first section in its active configuration having a diameter substantially equal to the first diameter; the second section in the nominal configuration having a diameter less than the second diameter and the second section in the active configuration having a diameter substantially equal to the second diameter; the third section in the nominal configuration having a diameter less than that of the fistula and the third section in the active configuration having a diameter substantially equal to that of the fistula.

17. The device of claim 13, further comprising a stabilizing portion, adjacent to a distal end of the second section, being manipulable between a nominal configuration and an active configuration, the nominal configuration having a diameter which is less than the second diameter and the active configuration having a diameter that is substantially equal to the second diameter.

18. The device of claim 17, wherein the stabilizing portion is an expandable hoop.

19. The device of claim 17, wherein the stabilizing portion is a row of stent cells arranged so that a first cell in the row is coupled to a last cell in the row, forming a hoop.

20. The device of claim 17, wherein the stabilizing portion comprises a plurality of rows of stent cells arranged so that a first cell in each row is coupled to a last cell in each row, forming a tube.

21. The device of claim 13, wherein the expandable tubular stent is constructed at least partially of nitinol.

22. The device of claim 13, wherein the expandable tubular stent contains nitinol and is covered with an impermeable membrane.

23. The device of claim 17, wherein the stabilizing portion is metallic.

24. The device of claim 17 wherein the stabilizing portion contains nitinol.

25. A catheter system for piercing a first wall of a first blood vessel and a second wall of a second blood vessel to create a fistula between the blood vessels, the system comprising:
a first catheter having a distal end insertable to a position wherein the distal end is adjacent a site within the first blood vessel for the fistula, the first catheter including a piercing tool adjacent the distal end;
a second catheter having a distal end insertable to a position wherein the distal end is adjacent a site within the second blood vessel for the fistula, the second catheter including adjacent the distal end a receptor having a distal opening, a proximal end, and a guide surface disposed between the distal opening and the proximal end;
one or more magnets disposed on at least one of the catheters to draw the piercing tool along the guide surface of the receptor; and
an expandable tubular stent with a first section having a first radial strength, a second section having a second radial strength, and a third section, positioned between the first and second sections, having a third radial strength which is stronger than the first or second radial strengths and at least one bend, the tubular stent being insertable to a position wherein the first section extends through the first blood vessel to a position adjacent to the fistula, the third section extends through the fistula, and the second section extends from a position adjacent the fistula into the second blood vessel.

26. The device of claim 25, wherein the expandable tubular stent is constructed at least partially of nitinol.

27. The device of claim 25, wherein the expandable tubular stent contains nitinol and is covered with an impermeable membrane.

28. A catheter system for piercing a first wall of a first blood vessel and a second wall of a second blood vessel to create a fistula between the blood vessels, the system comprising:
a first catheter having a distal end insertable to a position wherein the distal end is adjacent a site within the first blood vessel for the fistula, the first catheter including a piercing tool adjacent the distal end;
a second catheter having a distal end insertable to a position wherein the distal end is adjacent a site within the second blood vessel for the fistula, the second catheter including adjacent the distal end a receptor having a distal opening, a proximal end, and a guide surface disposed between the distal opening and the proximal end;
one or more magnets disposed on at least one of the catheters to draw the piercing tool along the guide surface of the receptor;
an expandable tubular portion having a proximal end and a distal end, the expandable tubular portion being insertable to a position wherein it extends from the first vessel through the fistula into the second vessel, with the proximal end in the first blood vessel and the distal end in the second blood vessel; and
a stabilizing portion, adjacent to the distal end of the expandable tubular portion, being manipulable between a nominal configuration and an active configuration, the nominal configuration having a diameter which is less than the diameter of the second blood vessel and the active configuration having a diameter that is substantially equal to the diameter of the second blood vessel.

29. The device of claim 28 wherein the expandable tubular stent is constructed at least partially of nitinol.

30. The device of claim 28 wherein the expandable tubular stent contains nitinol and is covered with an impermeable membrane.

31. The device of claim 28 wherein the stabilizing portion is an expandable hoop.

32. The device of claim 28 wherein the stabilizing portion is a row of stent cells arranged so that a first cell in the row is coupled to a last cell in the row, forming a hoop.

33. The device of claim 28 wherein the stabilizing portion comprises a plurality of rows of stent cells arranged so that a first cell in each row is coupled to a last cell in each row, forming a tube.

34. The device of claim 28 wherein the expandable tubular portion includes a first section having a first radial strength, a second section having a second radial strength, and a third section, positioned between the first and second sections, having a third radial strength which is stronger than the first or second radial strengths and at least one bend, the third section extending through the fistula.
